(19) United States
(12) Patent Application Publication
(10) Pub. No.: US 2026/0298871 A1
(43) Pub. Date: Oct. 1, 2026

(54) SYSTEM AND METHOD FOR MONITORING SULFITE

(71) Applicant: Watgrid, Lda., Ílhavo (PT)

(72) Inventors: Guilherme Cruto Ala da Costa, Ílhavo (PT); Daniel Duarte, Ílhavo (PT); Lúcia Maria Botas Bilro, Gafanha Da Encarnação (PT); Rogério Nunes Nogueira, Aveiro, Beira Litoral (PT)

(21) Appl. No.: 19/576,514

(22) Filed: Mar. 24, 2026

Related U.S. Application Data
(60) Provisional application No. 63/777,405, filed on Mar. 25, 2025.
(60) Provisional application No. 63/777,845, filed on Mar. 26, 2025.

(51) Int. Cl. G01N 27/49; G01N 27/28; G01N 27/30; G01N 33/14

(52) U.S. Cl. CPC G01N 27/49; G01N 27/283; G01N 27/301; G01N 27/308; G01N 33/146

(57) ABSTRACT

This disclosure relates to electrochemical monitoring of sulfite concentration within a vessel, such as a wine barrel, using a barrel-insertable probe that performs sequential cyclic voltammetry and detects sulfite dosing events according to a derivative of a difference between voltammograms acquired at different times. The disclosed sulfite monitoring system integrates sequential cyclic voltammetry, differential processing, derivative-based dynamic oxidation region detection, and binary classification to detect sulfite concentration variation events. Upon detection of a sulfite variation event, a calibration parameter is adaptively updated in a closed-loop configuration to improve robustness against matrix drift and peak shifting.

### PRIORITY CLAIM

This patent application claims the benefit of priority to U.S. provisional patent application 63/777,405, titled “System and Method for Monitoring Sulfite,” filed on Mar. 25, 2025 and U.S. provisional patent application 63/777,845, titled “System and Method for Monitoring Sulfite,” filed on Mar. 26, 2025. The above referenced documents are hereby incorporated herein by reference in their entirety.

### BACKGROUND

Limitations and disadvantages of conventional and traditional monitoring systems for winemaking will become apparent to one of skill in the art, through comparison of such systems with some aspects of the present invention as set forth in the remainder of the present application with reference to the drawings.

### BRIEF SUMMARY OF THE INVENTION

Monitoring systems for winemaking substantially as shown in and/or described in connection with at least one of the figures, as set forth more completely in the claims. These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 illustrates a barrel-insertable sulfite monitoring device in accordance with an example implementation of this disclosure.

FIG. 2 illustrates an electrochemical cell configuration in accordance with an example implementation of this disclosure.

FIG. 3 illustrates a cyclic voltammogram in accordance with an example implementation of this disclosure.

FIG. 4A illustrates an example difference signal and an example derivative of that difference signal when there is no significant sulfite variation in the wine sample.

FIG. 4B illustrates an example difference signal and an example derivative of that difference signal when there is a detectable sulfite variation in the wine sample.

FIG. 5 illustrates an example processing architecture in accordance with an example implementation of this disclosure.

### DETAILED DESCRIPTION OF THE INVENTION

This disclosure relates to electrochemical monitoring of sulfite concentration within a vessel, such as a wine barrel, using a barrel-insertable probe that performs sequential cyclic voltammetry and detects sulfite dosing events according to a derivative of a difference between voltammograms acquired at different times.

The disclosed system improves electrochemical sulfite monitoring by reducing baseline drift, electrode fouling effects, and matrix variability through temporal signal differencing and derivative enhancement performed while the sensing assembly remains installed within the vessel.

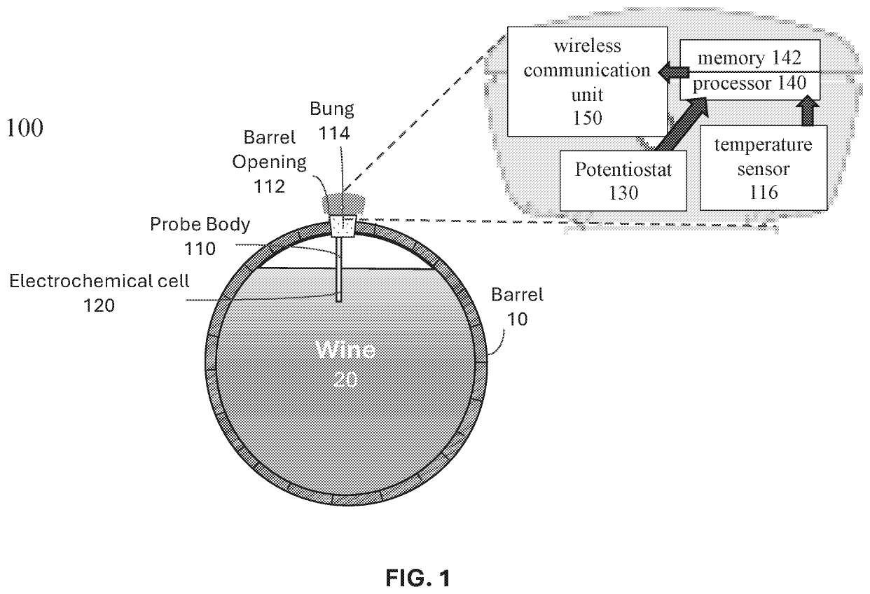

Referring to FIG. 1, sulfite monitoring device 100 is configured for insertion into barrel 10 containing wine 20.

Device 100 comprises elongated probe body 110 dimensioned to extend through barrel opening 112 and into wine 20. Probe body 110 may comprise stainless steel, food-grade polymer, or other chemically resistant material suitable for prolonged exposure to wine and fermentation gases.

Sealing structure 114 engages barrel opening 112 to maintain a liquid-tight and gas-controlled interface. Sealing structure 114 may comprise an elastomeric bung interface, compression seal, threaded collar, tapered plug, or expandable sealing member. The sealing structure maintains barrel integrity during monitoring and permits extended in situ operation.

Probe body 110 houses electrochemical cell 120 at a distal portion immersed in wine 20.

Device 100 may also comprise potentiostat 130, power supply 134, processor 140, memory 142, wireless communication unit 150, and temperature sensor 116.

Potentiostat 130 applies controlled voltage waveforms and measures resulting current responses. Processor 140 executes signal acquisition and transformation instructions stored in memory 142. Wireless communication unit 150 transmits event notifications or logged data to a remote system. Temperature sensor 116 provides temperature measurements for electrochemical compensation and monitoring of vinification processes, such as fermentation.

Device 100 is configured to remain inserted in barrel 10 for repeated measurements without removal of wine 20 and without addition of titration reagents. Processor 140 controls potentiostat 130 to perform cyclic voltammetry at repeated time intervals while device 100 remains installed in barrel 10.

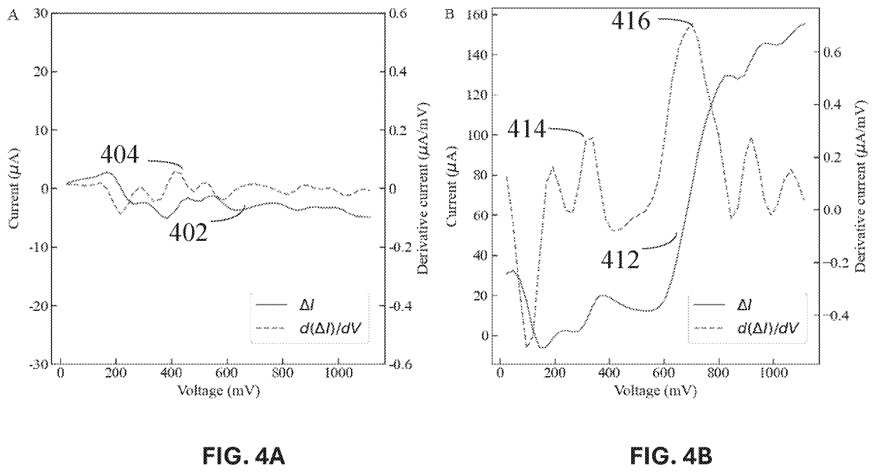

Device 100 may perform cyclic voltammetry at configurable intervals during vinification processes, such as fermentation and/or aging. Processor 140 may store, in memory 142, time-stamped records corresponding to detected sulfite dosing peaks 416 (described below in reference to FIG. 4B), thereby generating a chronological sulfite event history associated with barrel 10. Wireless communication unit 150 may transmit the stored event records to a remote winery monitoring system.

Temperature sensor 116 may provide compensation for temperature-dependent electrochemical behavior.

Because detection is performed in situ, device 100 eliminates the need for removal of wine samples and eliminates reliance on iodine titration, hydrogen peroxide oxidation, or colorimetric endpoint detection.

The disclosed system differs from titration-based sulfite testing methods in that electrochemical interrogation occurs continuously within barrel 10, no chemical reagents are introduced into wine 20, no manual sampling is required, and detection is based on temporal electrochemical change rather than visual endpoint.

The combination of barrel-insertable probe architecture and derivative-of-difference signal transformation provides improved sensitivity to sulfite dosing events during vinification processes, such as fermentation and/or aging.

Voltage sweep range may extend between approximately −1200 mV and +1200 mV relative to reference electrode 220. Scan rate may range between approximately 50 mV/s and 200 mV/s.

Power supply 134 may comprise an internal battery or external supply. Wireless communication unit 150 may utilize Bluetooth, Wi-Fi, cellular, or low-power wide-area communication protocols.

Electrochemical cell 200 may be adapted for use in stainless steel tanks or other vessels in addition to barrel 10.

In certain embodiments, device 100 may additionally perform pulsed amperometric detection to estimate absolute sulfite concentration, although detection of dosing events does not require reagent-based chemistry. In certain embodiments, absolute sulfite concentration is estimated using a calibration curve derived from pulsed amperometric detection (PAD). The calibration curve may be generated by fitting stabilized current parameters to known sulfite concentrations using regression models. In one implementation, a non-linear model of the form I=a·e^{-b·n}+c is fitted across PAD cycles, and parameter c is correlated to sulfite concentration via a first-degree or higher-order regression curve. In certain embodiments, when the classifier indicates no sulfite variation but a baseline shift is detected, the calibration curve is automatically adjusted. Adjustment may comprise an additive offset correction to compensate for drift, temperature variation, electrode aging, or matrix effects. In at least one implementation, such calibration adjustment is automatic and executed without user intervention.

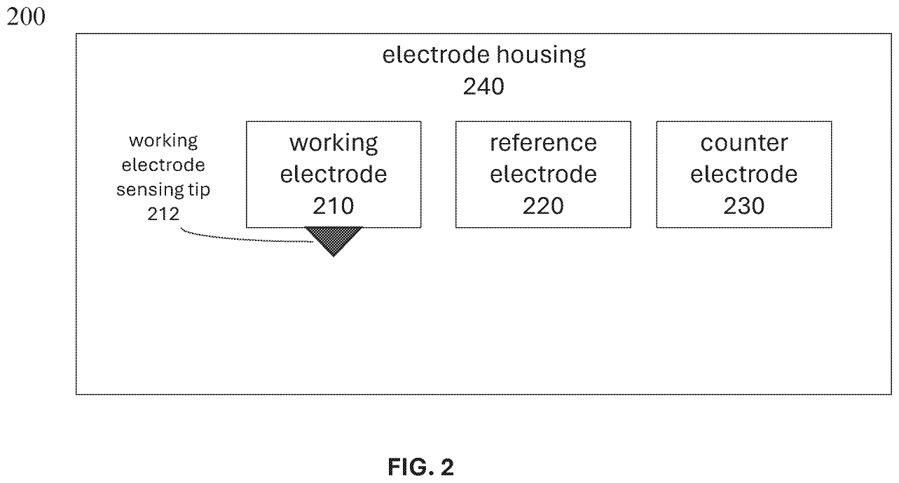

Referring to FIG. 2, electrochemical cell 200 comprises working electrode 210, working electrode sensing tip 212, reference electrode 220, counter electrode 230, and electrode housing 240.

Working electrode 210 defines sensing tip 212 and may comprise gold, platinum, glassy carbon, boron-doped diamond, or metalloporphyrin-modified carbon. Reference electrode 220 provides a stable reference potential. Counter electrode 230 completes the electrochemical circuit.

Electrode housing 240 mechanically supports electrodes 210, 220, and 230 while electrically isolating them from probe body 110 (of FIG. 1). Electrode spacing and exposure geometry may be selected to maintain stable diffusion conditions during cyclic voltammetry.

Potentiostat 130 (of FIG. 1) applies a cyclic voltage waveform to working electrode 210 relative to reference electrode 220 and measures current between working electrode 210 and counter electrode 230.

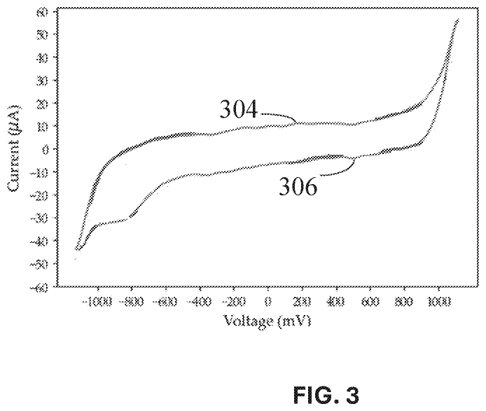

FIG. 3 illustrates a cyclic voltammogram in accordance with an example implementation of this disclosure. Each cyclic voltammogram comprises forward scan region 304 and reverse scan region 306. Forward scan region 304 corresponds to increasing applied potential. Reverse scan region 306 corresponds to decreasing applied potential.

At a first time t₁, a first cyclic voltammogram is acquired and represented as I₁(V). At a second time t₂, a second cyclic voltammogram is acquired and represented as I₂(V). The time interval between t₁ and t₂ may range from minutes to hours depending on vinification processes, such as fermentation and/or aging.

Because electrochemical baseline drift, electrode conditioning effects, and matrix composition may evolve slowly over time, direct comparison of sequential voltammograms provides improved sensitivity to chemical change relative to absolute peak measurement alone. In certain implementations, the derivative is computed on the difference signal according to d(ΔI(V))/dV, where ΔI(V)=I₂(V)−I₁(V). Although mathematically equivalent to computing the difference between derivatives of sequential voltammograms, performing differentiation after formation of the difference signal may improve suppression of slow baseline drift and electrode conditioning effects prior to derivative enhancement.

Processor 140 (as shown in FIG. 1) generates difference signals according to:

$$Δ I ( V ) = {I}_{2} ( V ) - {I}_{1} ( V )$$

and corresponding derivative signals according to:

$$d ( Δ I ) / dV$$

In certain embodiments, derivative analysis is performed within a selected oxidation potential window corresponding to sulfite electrochemical activity. For example, analysis may be restricted to a voltage range between approximately −1.2 V and +1.2 V relative to the reference electrode. Limiting analysis to the oxidation profile reduces influence from reduction-side phenomena and non-relevant electrochemical processes. In alternative implementations, different voltage windows may be selected depending on electrode material or analyte chemistry.

Because a cyclic voltammogram comprises a forward component 304 and a reverse component 306, the difference signal and derivative signal may contain corresponding both forward and reverse components. Alternatively, the forward and reverse components may be averaged or only one of the forward and reverse components may be used.

FIG. 4A illustrates an example difference signal 402 and an example derivative 404 of that difference signal when there is no significant sulfite variation in the wine sample. Generation of ΔI(V) reduces stable baseline current components and suppresses slow electrode drift, thereby isolating electrochemical changes that occurred between t₁ and t₂.

FIG. 4B illustrates an example difference signal 412 and an example derivative 414 of that difference signal when there is a detectable sulfite variation in the wine sample. If sulfite concentration remains unchanged between t₁ and t₂, difference signal 402 remains near zero across the oxidation region.

A shown in FIG. 4B, derivative signal d(ΔI)/dV enhances inflection features associated with sulfite oxidation kinetics and sharpens discrimination of change events. Derivative signal 414 comprises sulfite dosing peak 416.

Sulfite dosing peak 416 corresponds to a local maximum in derivative signal 414 and indicates that a sulfite concentration change occurred between t₁ and t₂. In certain embodiments, the derivative signal within the selected voltage window is subjected to dimensionality reduction prior to classification or event determination. Dimensionality reduction may comprise principal component analysis (PCA), singular value decomposition (SVD), linear discriminant analysis (LDA), autoencoder compression, or equivalent feature extraction techniques. In at least one implementation, dimensionality reduction is required to reduce noise sensitivity and to extract compact feature vectors representative of sulfite-related electrochemical behavior. In certain implementations, the reduced feature vector is evaluated within a binary framework configured to distinguish between (i) no sulfite variation and (ii) occurrence of sulfite variation between measurement times t₁ and t₂. In an initial deployment configuration, this binary framework operates as a labeling system used for validation and system verification. In other embodiments, the binary decision logic may comprise threshold rules, support vector machine (SVM) boundaries, Gaussian mixture models (GMM), neural network classifiers, probabilistic hypothesis testing, or equivalent classification mechanisms. In certain embodiments, the classifier is incorporated into the operational sensing loop and directly influences calibration or reported sulfite values. In alternative embodiments, including early deployment configurations, the classifier is used for validation and performance assessment without directly modifying reported concentration outputs.

Derivative computation may be performed using finite difference methods, polynomial fitting, smoothing-based differentiation, or equivalent numerical techniques.

By operating on ΔI(V) rather than on a single voltammogram, the derivative emphasizes change-based electrochemical behavior and reduces sensitivity to absolute baseline magnitude.

Detection of sulfite dosing peak 416 constitutes detection of a sulfite dosing event while probe body 110 remains positioned within barrel 10 as illustrated in FIG. 1.

If sulfite is introduced or depleted between t₁ and t₂, difference signal 412 exhibits characteristic lobes corresponding to altered oxidation current.

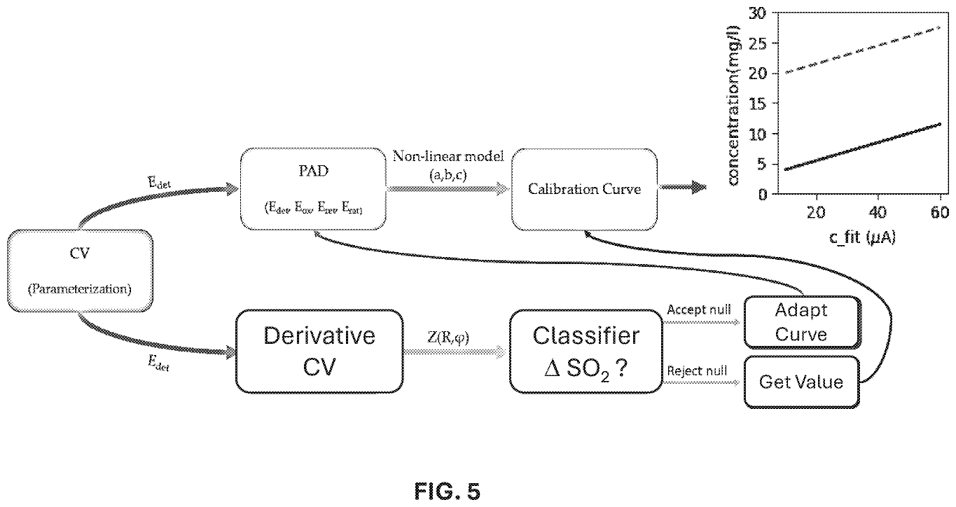

Referring to FIG. 5, a processing architecture is illustrated in which derivative cyclic voltammetry is integrated with classification and calibration control. Sequential cyclic voltammograms are acquired and transformed into a derivative-of-difference representation within the selected oxidation window. The resulting feature set is provided to a classifier configured to determine whether a change in sulfite concentration (ΔSO₂) has occurred.

If a sulfite variation event is detected, the system reports the updated concentration value. If no sulfite variation is detected but baseline shift is observed, the system automatically adapts the calibration curve using PAD-derived parameters to maintain concentration accuracy. This closed-loop structure enables both event detection and long-term drift compensation during in situ operation within a vessel.

This temporal differencing constitutes a physical signal conditioning improvement applied to electrochemical data acquired within the barrel.

The foregoing description illustrates representative embodiments. Variations in electrode materials, probe geometry, sealing mechanisms, communication interfaces, and power configurations may be implemented without departing from the scope of the appended claims.

As utilized herein the terms “circuits” and “circuitry” refer to physical electronic components (i.e. hardware) and any software and/or firmware (“code”) which may configure the hardware, be executed by the hardware, and or otherwise be associated with the hardware. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled or not enabled (e.g., by a user-configurable setting, factory trim, etc.).

The present invention may be realized in hardware, software, or a combination of hardware and software. The present invention may be realized in a centralized fashion in at least one computing system or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

The present invention may be realized in a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

While the present invention has been described with reference to certain embodiments, it will be understood by those skilled in the art that various changes may be made and equivalents may be substituted without departing from the scope of the present invention. Those skilled in the art will understand that, in addition to winemaking, the present invention may be used for making of other products (e.g., whiskey, cognac, brandy, rum, gin, vodka, tequila, beer) without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A sulfite monitoring device configured for insertion into a vessel containing wine, comprising:
an elongated probe body dimensioned to extend through an opening of the vessel and into wine contained therein;
a sealing structure configured to engage the opening of the vessel;
an electrochemical cell disposed within the probe body and comprising a working electrode, a reference electrode, and a counter electrode exposed to the wine;
a potentiostat configured to apply a cyclic voltage waveform to the working electrode relative to the reference electrode and measure current between the working electrode and the counter electrode; and
a processor configured to:
acquire a first cyclic voltammogram at a first time and a second cyclic voltammogram at a second time while the probe body remains inserted in the vessel,
generate a difference signal according to ΔI(V)=I₂(V)−I₁(V),
generate a derivative of the difference signal with respect to applied potential,
extract, from the derivative within a selected oxidation potential window, a feature representation corresponding to electrochemical change between the first time and the second time,
apply dimensionality reduction to the feature representation to generate a reduced feature vector,
evaluate the reduced feature vector using a classifier configured to determine whether a sulfite concentration variation has occurred, and
in response to the classifier determination:
detect a sulfite dosing event when a sulfite concentration variation is determined, and
automatically adjust a calibration parameter when no sulfite concentration variation is determined and a baseline shift is detected.

2. The device of claim 1, wherein the vessel comprises a wine barrel and the sealing structure comprises a bung interface configured to maintain barrel integrity.

3. The device of claim 1, wherein the working electrode comprises gold.

4. The device of claim 1, wherein the working electrode comprises metalloporphyrin-modified carbon.

5. The device of claim 1, wherein the cyclic voltage waveform comprises a forward scan region and a reverse scan region.

6. The device of claim 5, wherein the difference signal comprises a forward scan difference component and a reverse scan difference component.

7. The device of claim 1, wherein the processor detects the sulfite dosing event according to a local maximum in the derivative of the difference signal.

8. The device of claim 1, wherein the processor suppresses baseline drift by subtracting the first cyclic voltammogram from the second cyclic voltammogram prior to derivative computation.

9. The device of claim 1, comprising a temperature sensor disposed within the probe body and configured to provide temperature data associated with the wine.

10. The device of claim 1, comprising a wireless communication unit configured to transmit sulfite dosing event data to a remote monitoring system.

11. A method of detecting sulfite concentration change within wine contained in a vessel, comprising:
inserting an electrochemical probe into the vessel such that a working electrode, a reference electrode, and a counter electrode are exposed to the wine;
applying a cyclic voltage waveform to the working electrode relative to the reference electrode;
acquiring a first cyclic voltammogram at a first time and a second cyclic voltammogram at a second time while the probe remains inserted in the vessel;
generating a difference signal according to ΔI(V)=I₂(V)−I₁(V);
computing a derivative of the difference signal with respect to applied potential;
extracting, from the derivative within a selected oxidation potential window, a feature representation corresponding to electrochemical change;
applying dimensionality reduction to the feature representation to generate a reduced feature vector;
classifying the reduced feature vector to determine whether a sulfite concentration variation has occurred; and
in response to the classification:
detecting a sulfite dosing event when sulfite variation is determined, and automatically adjusting a calibration parameter when sulfite variation is not determined and a baseline shift is detected.

12. The method of claim 11, wherein the vessel comprises a wine barrel.

13. The method of claim 11, wherein the cyclic voltage waveform comprises a forward scan and a reverse scan.

14. The method of claim 13, wherein the difference signal includes forward and reverse scan components.

15. The method of claim 11, wherein the derivative is computed using a finite difference technique.

16. The method of claim 11, wherein detection of the sulfite dosing event is performed without removal of wine from the vessel.

17. The method of claim 11, wherein the probe remains installed in the vessel during repeated cyclic voltammetry measurements over a predetermined period.

18. The method of claim 11, comprising transmitting sulfite dosing event data wirelessly to a remote system.

19. The method of claim 11, comprising measuring wine temperature and associating the measured temperature with the cyclic voltammetry data.

20. The method of claim 11, wherein the cyclic voltage waveform extends between approximately −1200 mV and +1200 mV relative to the reference electrode.
